(19) United States
(12) Patent Application Publication
(10) Pub. No.: US 2023/0197259 A1
(43) Pub. Date: Jun. 22, 2023

(54) GENERATING A RECOMMENDED HEALTHCARE DECISION PROCESS

(71) Applicant: KONINKLIJKE PHILIPS N.V., Eindhoven (NL)

(72) Inventors: Nathalie Magali Danielle Dessaud, Eindhoven (NL); Martine Huygens, Eindhoven (NL)

(21) Appl. No.: 18/077,278

(22) Filed: Dec. 8, 2022

Related U.S. Application Data
(60) Provisional application No. 63/291,504, filed on Dec. 20, 2021.

(51) Int. Cl. G16H 40/20

(52) U.S. Cl. CPC G16H 40/20

(57) ABSTRACT

A mechanism for controlling or defining a decision guidance process. A decision guidance process guides a subject and/or caregiver (of the subject) through a decision making process in making a clinical or healthcare decision. The proposed mechanism makes use of motivation data to control the decision guidance process.

### BACKGROUND OF THE INVENTION

### 1. Field of the Invention

The present invention relates to the field of healthcare, and in particular, to the generation of a recommended approach for making a healthcare decision.

### 2. Description of the Related Art

A key ambition of healthcare providers is to ensure that patients/subjects receive appropriate healthcare that reduces their risks of more serious conditions and improves their quality of life and likely health outcome. This can be achieved through appropriate decision making processes for future healthcare of the subject. The process of making a healthcare decision is often called a conversion, as the subject is converted to a particular healthcare decision as to which treatment or solution they are to pursue.

One ongoing problem with the decision making process, or conversion, is that a decision may not happen in a timely manner. This can be due to the difficulty of identifying symptoms, getting a diagnosis, the load of the healthcare system, a fear of overloading the (healthcare) system and/or concerns over potential costs. There are also ongoing concerns that an inappropriate decision making process or conversion will lead to an incorrect healthcare decision (i.e., not selecting an appropriate treatment or solution), which could lead to a subject not receiving the healthcare they require.

As such, there has been an increasing interest in decision guidance processes that guide or aid a clinician and/or the patient in making a decision about the patient's/subject's future healthcare, i.e., aid the conversion process.

There is an ongoing need to provide approaches that facilitate improved speed, efficiency, accuracy and effectiveness of the decision making process. There is a clear and direct medical advantage of such an improved approach, as appropriate guidance results in improved decision making processes, which increase a likelihood that a subject receives suitable medical care for their goals, thereby improving their quality of life and health outcomes.

### SUMMARY OF THE INVENTION

According to examples in accordance with an aspect of the invention, there is provided a computer-implemented method of controlling a decision guidance process for guiding a subject and/or caregiver in a decision making process for making a healthcare decision for the subject.

The computer-implemented method comprises: determining motivation data of the subject, wherein the motivation data indicates one or more predicted measures of the subject's motivation for the subject's future healthcare; and controlling the decision guidance process responsive to the determined motivation data of the subject.

The proposed approach recognizes that the likely adherence of a subject to a future healthcare is dependent upon the motivation of the subject to carry out the healthcare plan or about their future healthcare. It is herein established that there is a link between motivation and the decision making process, such that controlling the decision guidance process (and therefore the decision making process) can control a likely success of future healthcare.

For instance, an extremely long decision making process or an information heavy decision guidance process is likely to demotivate an already highly motivated subject, e.g., by causing dissatisfaction or through delay. There is also a risk that an extremely long decision making process will causes a subject/patient to doubt or second-guess any decisions that they make, leading to a reduced likelihood that they will adhere to or follow a future healthcare plan.

On the other hand, it is similarly herein recognized that patients who are not initially motivated to engage in their future healthcare would benefit from a longer decision making process and/or additional information during the decision making process. This may give an opportunity to increase the subject's motivation, e.g., by providing them with an opportunity to reflect and/or understand their options. Increasing their motivation before beginning future healthcare may avoid a subject not starting their future healthcare or dropping out at an early stage (as has been observed with unmotivated subjects), which leads to reduced quality of life and adverse medical outcomes.

Based on at least this realization, it is proposed to control a decision guidance process (that guides a subject and/or caregiver through the decision making process) based on one or more predicted measures of motivation for the subject.

The proposed approach to controlling the decision guidance process thereby facilitates an increased likelihood that a subject will adhere to future healthcare following the decision making process. In particular, this approach may make sure that subjects are committed to, i.e., motivated to stick to, the care path that best suits their needs.

In some examples, the decision guidance process comprises providing or displaying guidance to the subject and/or caregiver for aiding in the performance of a decision making process, e.g., recommending next steps in a decision making process or providing an indication of the overall decision making process (e.g., aims or targets). The proposed approach provides a mechanism for controlling the decision making process, e.g., to control what information is provided or displayed to the subject as a result of the decision guidance process.

The step of controlling the decision guidance process may comprise controlling a length of the decision guidance process responsive to the determined motivation data. This embodiment recognizes that a length of the decision guidance process, i.e., how long the decision guidance process gives the subject/clinician to make the clinical decision, has a significant effect on the motivation of the subject for their future healthcare. Thus, by controlling the length of the decision guidance process, a likelihood that a subject will adhere to the decided cause of action for their healthcare is increased.

In at least one example, the step of controlling a length of the decision guidance process comprises controlling a length of a delay of guidance to be provided to the subject and/or caregiver. This provides a simple and effective mechanism for modulating or controlling the speed of the decision making process.

As another example, the step of controlling a length of the decision guidance process may comprise controlling a speed at which guidance is provided to the subject and/or caregiver.

In some embodiments, the decision guidance process defines one or more recommended steps for the subject and/or caregiver to take in the decision making process; and the step of controlling the decision guidance process comprises controlling the defining of the one or more recommended steps responsive to the motivation data.

In this way, the (content, number and/or type of) decision making steps that are recommended to the subject may be based upon the subject's motivation. This provides an approach for tailoring or customizing the recommended decision making steps for the subject, to improve a likelihood that the subject will make an appropriate healthcare decision and/or adhere to the outcome of a healthcare decision.

Optionally, the step of controlling the decision guidance process comprises controlling whether or not a step, for the decision making process, is included in the one or more recommended steps. Removing or adding recommended steps provides one approach for controlling the speed of the decision making process. Moreover, a decision can be made as to whether a particular step is desired or required for aiding the subject or caregiver in making a decision. For instance, a highly motivated subject may not require a step of “consider your options overnight”, as they may already be confident on a cause of action.

The step of controlling the decision guidance process may comprise selecting a recommended step based on a predicted length of time for the subject and/or caregiver to carry out the recommended step. This embodiment provides an approach for controlling the length of a decision making process, which has an influence on the motivation of the subject and therefore the likelihood of a subject to carry out an outcome of a healthcare decision.

In some examples, the decision guidance process defines a sequence of stages for the decision making process, wherein each stage is able to be completed using one of a respective set of steps; and the step of controlling the decision guidance process comprises selecting, for each stage, a step from the respective set of steps to be carried out by the subject and/or caregiver. In particular, a single step from the respective set of steps may be selected for the clinician/caregiver to carry out to perform the stage of the decision making process.

This approach means that alternative steps may be chosen to perform a certain stage of the decision making process, which facilitates control over the speed of the decision making process as well as the content of the decision guidance process. This facilitates customization of the decision guidance process for a subject.

The computer-implemented method may further comprise a step of determining urgency data of the subject, wherein the urgency data indicates one or more predicted measures of the subject's urgency for receiving future healthcare; and wherein the step of controlling the decision guidance process comprises controlling the decision guidance process to be further responsive to the determined urgency data.

The present disclosure also recognizes that an urgency of a subject for receiving healthcare is important in controlling the decision guidance process. More urgent cases may require a quicker decision making process than less urgent cases to reduce an impact or progression of a particular pathology. In some examples, the speed or length of the decision guidance process may be controlled responsive to the urgency of the subject.

The step of controlling the decision guidance process may comprise: using the motivation data and the urgency data to determine a conversion readiness level, indicating a readiness of the subject and/or caregiver to make a healthcare decision; and controlling the decision guidance process responsive to the conversion readiness level.

The method may further comprise a step of determining resource data for the subject, wherein the resource data indicates one or more predicted measures of the availability of a healthcare resource for the subject during the subject's future healthcare, wherein the step of controlling the decision guidance process comprises controlling the decision guidance process to be further responsive to the determined resource data.

Optionally, the step of determining motivation data of the subject comprises: tracking the actions of the subject and/or caregiver during the decision making process; and using the tracked actions to determine the motivation data of the subject.

This approach provides a direct mechanism for assessing the motivation of the subject with respect to their future healthcare. In particular, it has been recognized that the actions of the subject during the decision making process reflect the likely motivation of the subject. For instance, an unmotivated subject is unlikely to adhere to the decision making process and/or take a long time to perform recommended steps for the decision making process.

In some examples, the decision guidance process defines one or more recommended steps for the subject and/or caregiver to take in the decision making process; and the step of tracking the actions of the subject comprises tracking completion of each recommended step by the subject and/or caregiver.

In some examples, the step of tracking the actions of the subject and/or caregiver comprises tracking a length of time required to perform each recommended step by the subject and/or caregiver.

There is also proposed a computer program product comprising computer program code means which, when executed on a computing device having a processing system, cause the processing system to perform all of the steps of the method according to any herein described method.

There is also proposed a processing system configured to control a decision guidance process for guiding a subject and/or caregiver in a decision making process for making a healthcare decision for the subject, the processing system being configured to: determine motivation data of the subject, wherein the motivation data indicates one or more predicted measures of the subject's motivation for the subject's future healthcare; and control the decision guidance process responsive to the determined motivation data of the subject.

Any herein described processing system may be configured to carry out the steps of any herein described method, and vice versa. These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a processing system according to an embodiment;

FIG. 2 illustrates a method according to an embodiment; and

FIG. 3 illustrates a processing system according to an embodiment.

### DETAILED DESCRIPTION OF THE EMBODIMENTS

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a mechanism for controlling or defining a decision guidance process. A decision guidance process guides a subject and/or caregiver (of the subject) through a decision making process in making a clinical or healthcare decision. The proposed mechanism makes use of motivation data to control the decision guidance process.

Embodiments are based on the realization that the characteristics, e.g., content or length, of a decision guidance process is likely to affect a motivation of a subject to make or adhere to a healthcare decision. By controlling a decision guidance process based on the motivation of the subject, the motivation levels of the subject can be controlled such that there is an increased likelihood that the subject will make and stick to a healthcare decision.

Approaches described in the present disclosure may be employed in any suitable healthcare or clinical scenario, in which a healthcare decision needs to be made by a subject or caregiver.

In the context of the present disclosure, the term “patient” is considered to be interchangeable with the term “subject”.

The present disclosure provides approaches for improving the conversion of patients to a healthcare solution. These approach aim to solve problems faced by both patients and healthcare providers. For instance, being too successful in conversion can lead to overload in healthcare systems, which will subsequently reduce patient satisfaction, motivation and engagement if patients need to wait to get a medical solution (e.g., a doctor visit, a diagnosis, a new therapy etc.) for any health problems they are worried about.

Compared to conventional methods based on healthcare needs solely, the present disclosure recognizes a need to convert people with the right level of motivation to ensure they are committed to the care path they need. This will prevent losing them in a healthcare path because of dissatisfaction and demotivation due to longer conversion times. On the other side, for patients not initially “motivated” to engage in a healthcare path, there is a need to increase their motivation first to prevent them from not starting or dropping out too early.

The proposed mechanism facilitates a decision guidance process for converting a subject to a healthcare decision, e.g., guiding them through a healthcare decision process, whilst keeping them engaged and motivated in their healthcare outcome. This is achieved by modifying the decision guidance process based on one or more estimated/predicted measures of their motivation for their future healthcare. Further embodiments control the decision guidance process based on an urgency of the subject's healthcare and available resources for providing a medical solution (both of which impact a speed at which care can or should be provided).

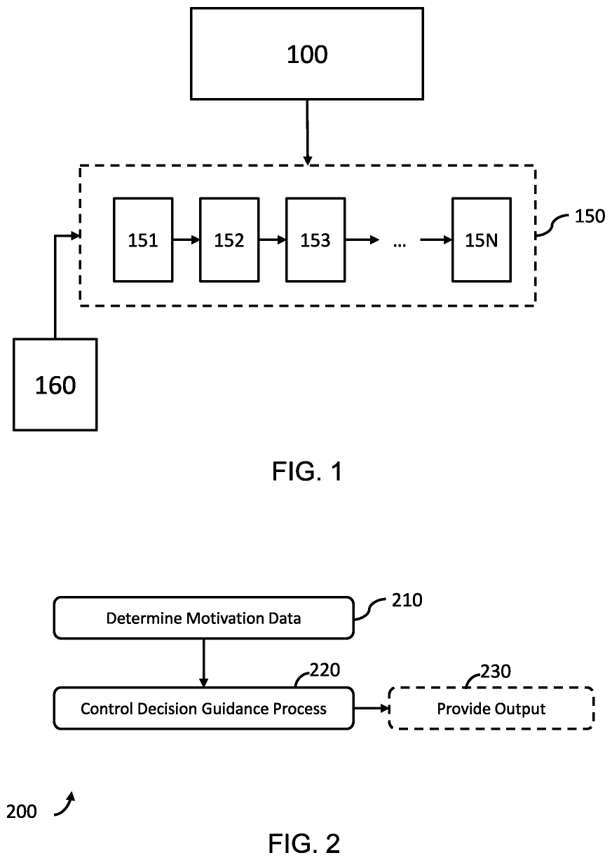

FIG. 1 conceptually illustrates an overview of a processing system 100 for controlling a decision guidance process 150.

A decision guidance process 150 provides a series or sequence of features 151, 152, 153 . . . 15N to a user. Each feature represents the provision of information or guidance to a subject to guide them through a particular phase/step of a decision making process. Each feature may, in particular, provide information to guide or aid the subject/caregiver in performing a particular phase of the decision making process. In particular, each feature may define a recommend step for the subject and/or caregiver to take in the decision making process.

For instance, one feature may provide diagnosis information, a second may advise to reflect upon diagnosis information and a third may be to provide information for understanding diagnosis information. Yet another feature may be to provide one or more possible treatment options, with a further feature comprising advising to reflect upon the different treatment options. Other suitable examples of features for aiding in a step of making a clinical or treatment decision will be apparent to the skilled person.

For instance, other examples of suitable features include: making, or providing a recommendation to make, an appointment with one or more caregivers/specialists; or requesting one or more measures of signs/symptoms (e.g., vital measurements such as blood pressure or blood glucose and/or self-reporting symptoms) as a single point measurement or over the course of a time period, e.g., few days. Another example of a feature may be to plan or recommend a laboratory test for the subject, e.g., to obtain additional information about the subject, e.g., measure HbA1C, pH levels, (certain) bacteria, blood gas, chromosome, c-reactive protein, electrolyte, ESR and/or cholesterol levels.

By way of further explanation, a decision making process (which is performed by a subject and/or caregiver) can be conceptually divided into a series of phases through which the subject and/or caregiver progresses in order to make a clinical decision (i.e., a healthcare decision). The decision guidance process 150 provides, for each phase, information, suggestions or guidance to the subject/caregiver to aid them in carrying out that phase of the decision making process. In this way, the decision guidance process provides a sequence of features 151, 152, 153 . . . 15N (i.e., information or guidance) to guide the subject/caregiver through the decision making process.

In this way, the decision guidance process may effectively define a sequence of stages/phases for the decision making process, e.g., wherein the features provide information or guidance for performing each stage. Each stage able to be completed using one of a respective set of steps (i.e., different versions of the feature provided by the decision guidance process).

Providing a feature to a caregiver or subject may comprise providing a user-perceptible output of the feature, e.g., in the form of a visual and/or audio representation of the feature. In this way, information for aiding or guiding a subject/caregiver in performing a phase of a decision making process (i.e., a feature) can be provided in the form of a user-perceptible output.

The decision guidance process 150 may have been originally generated (e.g., by a decision guidance generator 160) based on subject health information. For instance, health information of the subject may be processed in order to generate a decision guidance process that guides a subject/caregiver through a decision making process to direct them to one or more healthcare solutions.

As one example, the decision guidance generator 160 may identify one or more similar subjects (to the current subject) based on the subject health information. The decision guidance process 150 may then be generated based on the (preferably: previously successful) decision making process performed by the one or more similar subjects. For instance, features of the decision guidance process may provide information on the steps performed by the one or more similar subjects. As a further example, the features of the decision guidance process may be based upon the most commonly performed step(s) of the decision making process.

A “similar subject” may be a subject who shares one or more characteristics with the target subject, e.g., one or more demographic characteristics, diagnosis information, health care payer claims based data, heathcare utilisation, social determinants of health data. Other approaches for identifying similar subjects will be apparent to the skilled person, e.g., using clustering algorithms or the like.

As another example, a database may define recommended decision making processes for subjects having certain health conditions (e.g., different processes may be used for deciding a treatment for different types of cancer) and optionally certain motivation levels (which are discussed later in this disclosure). The decision guidance generator 160 may provide information for guiding a subject in performing the recommended decision making process associated with the health information of the subject.

For instance, the decision guidance generator 160 may obtain health information from a variety of sources, such as a user interface, monitoring devices (e.g., heart rate monitors or ECG monitors) and/or a memory unit. The health information may comprise, for instance, information from an electronic medical record (e.g., co-morbidity, diagnoses, demographic information, medical history, family medical history, urgency level), from monitoring devices (e.g., a heart rate monitor, blood pressure monitor, an ECG monitor, a blood glucose monitor, an AFib detection device, a scale); and/or a user interface (e.g., that provides self-reported complains and/or symptoms).

The decision guidance process 150 may provide one or more alternative versions of a feature to be provided to the subject/caregiver. One of these versions may act as a “default” version (which is used in the absence of any controlling mechanism).

The processing system 100 controls the decision guidance process, and may control an already defined or generated decision guidance process.

In a hereafter described embodiment, the processing system 100 controls the length of the decision guidance process 150 (and thereby the length of the decision making process). In this specific embodiment, the processing system 100 controls, for each feature, the length of time that the feature is provided to the subject and/or caregiver. Other examples for controlling the decision guidance process are later described.

Motivation data of the subject is used by the processing system 100 to control the decision guidance process. The motivation data may comprise a motivation level μ of the subject, which is calculated/evaluated by the processing system 100. The motivation level μ may be calculated based on one or more characteristics and/or information of the subject.

Examples include subject-reported information on motivation (e.g., a scalar indicator of their perceived motivation levels); one or more indicators of the subject's health literacy; one or more indicators of the subject's engagement with the decision making process (e.g., a measure of an amount of interaction with the decision guidance process), motivation data of similar subjects; historical motivation data of the subject (e.g., derived from previous decision making processes); a measure of self-efficacy; a measure of self-management; an indicator of the subject's anxiety about their health (e.g., a self-reported indicator) and/or a measure of social support available for the subject.

By way of example, a subject-reported indication of motivation may be provided at a user interface. Historic motivation data may be used if the current health information of the subject shares one or more similarities (e.g., severity or pathology type) with the health information of the subject at the time of the historic motivation data. Motivation data may also be derived from third party information, such as a user's interactions with social media or the like (e.g., the greater an interaction, the greater the motivation) and/or by natural language processing of data provided by the subject (e.g., “I can do this” indicating a higher level of motivation than “I can't do this”).

Other approaches for determining a motivation level could be used, such as those proposed by International Patent Application No. WO 2020/120596.

Information for determining a motivation data may be obtained (by the processing system) from a memory unit and/or a user interface.

If no information on a motivation level is available, then the motivation level may be assumed to have a default value (e.g., representing an average level of motivation for a population).

The motivation level may be formed of binary, categorical or numeric data. For instance, the motivation level may be a categorical indicator (e.g., “Low”, “Medium” or “High”) of the motivation of the subject. Thus, the motivation level may be effectively be a motivation state. As another example, the motivation level may be a numeric measure (e.g., on a scale of 0-1, 0-10, 0-100, 1-10 or 1-100, although other scales would be recognized by the skilled person).

The motivation level is used to control a length of time that each particular feature is provided to the subject/caregiver during the decision guidance process 150. In particular, a length of time may be controlled so that a predicted motivation level of the subject following completion of the phase of the decision making process (associated with the particular feature) is maintained and/or improved.

It is herein recognized that a length of time that a subject spends in a particular phase of a decision making process impacts the motivation of the subject at the end of that phase. For instance, if a highly motivated subject spends a long time on a particular phase, then they may lose motivation. On the contrary, if a subject with very low motivation spends a long time on a particular phase, then they may gain motivation as they are given time to consider their options.

In preferred examples, the processing system 100 increases the length of the decision guidance process responsive to the motivation data indicating that the subject has a low motivation (e.g., a classification of “Low” for a categorical data format or a value below a first predetermined value for a numeric measure of motivation). This corresponds with a prediction that a subject with a low level of motivation is predicted increase their motivation levels if more time is spent during (part of) a decision making process.

In preferred examples, the processing system 100 decreases the length of the decision guidance process responsive to the motivation data indicating that the subject has a high motivation (e.g., a classification of “high” for a categorical data format or a value above a second predetermined value for a numeric measure of motivation). This corresponds with a prediction that a subject with a high level of motivation is predicted to decrease their motivation levels if more time is spent during (part of) a decision making process.

In some examples, the processing system 100 maintains the length of the decision guidance process responsive to the motivation data indicating that the subject has a medium motivation (e.g., a classification of “Medium” for a categorical data format or a value between the first and second predetermined values for a numeric measure of motivation).

Other suitable approaches and techniques for controlling a length of the decision guidance process based on motivation data will be apparent to the skilled person.

The motivation level may be recalculated or re-evaluated after each phase of the decision making process, i.e., each time a feature is provided to the subject/caregiver by the decision guidance process. In this way, the decision guidance process is dynamic, responsive to an iteratively measured or determined motivation level.

Table 1 illustrates the predicted impact of a duration of a feature i on the motivation level of the subject (which is here a categorical indicator). This demonstrates the relationship between a motivation level of the subject and how the duration of the decision making process (or a stage of the decision making process) affects their future motivation levels.

TABLE 1
Predicted motivation states

| Motivation level | | Predicted Motivation |
|---|---|---|
| before Feature i | Duration | level After using |
| (L, M, H) μᵢ₋₁ | (L, M, H) | Feature i: μᵢ |
| Low | High | M |
| Low | Medium | M |
| Low | Low | L |
| Medium | High | L |
| Medium | Medium | H |
| Medium | Low | H |
| High | High | M |
| High | Medium | M |
| High | Low | H |

The length of a low, medium and high duration may depend upon the pathology about which a clinical decision is to be made and/or an urgency of the clinical decision. For instance, for an extremely urgent (or emergency) case, a low duration may be 1 minute; a medium duration may be 15 minutes and a high duration may be 1 hour. Similarly, for a medium urgency case, a low duration may be 1 hour, a medium duration may be 24 hours and a high duration may be 7 days. For a low urgency case, a low duration may be 1 day, a medium duration may be 30 days and a high duration may be 6 months.

The precise range may be defined based on acceptable clinical practice (e.g., to avoid a decision making process for an urgent case taking a medically-unsafe period of time) and/or may be self-learning.

Thus, in a first embodiment, the motivation level of the subject is determined at each phase of the decision making process (i.e., for each feature to be provided to the subject by the decision guidance process) and used to define the length of the next feature provided to the subject. In this way, the processing system controls the decision guidance process responsive to the determined motivation data of the subject.

Various approaches for controlling or defining the length of a feature are envisaged in this disclosure. Thus, the speed of a feature (or series of features) can be modulated based on several different techniques.

As one example, the processing system may control the decision guidance process to delay providing a next feature after a user provides some input (e.g., indicating completion of a previous phase of the decision making process or readiness to move to the next feature), immediately, several hours or days after (with or without message on expectation).

As another example, the processing system may control the decision guidance process to select one of multiple versions of a same feature (each expected to last different amounts of time). As an example a short feature may be: “Please select from the statement below which one are more important for you”, whereas a long feature may be “Tomorrow you will be asked to select from a list of statements that are relevant for you. Please take some time/the night to think about what is really important for you”. In this way, the subject/caregiver is provided with a variable length of time to complete the next phase of the decision making process.

As yet another example, the processing system may control the decision guidance process to add an additional or supplementary feature in the decision guidance process (and therefore an additional phase in the decision making process). For instance, if the processing system wishes to increase a length of the decision guidance process, a feature may be added for the subject/caregiver to monitor a particular sign/symptom over a period of time. This approach may increase the subject's motivation by providing them with an active action to take during the decision making process, and making the subject more aware of their signs/symptoms.

Of course, in some instances, the processing system may control the decision guidance process to remove one or more features of the decision guidance process in order to speed up the decision guidance process.

In some examples, each feature is associated with a particular priority (e.g., a measure of importance), which can be derived from historical examples of decision making processes performed by similar subject and/or the same subject. If a feature is to be removed, the removed feature may be based on a priority of the features in the decision guidance process (e.g., a lower priority feature may be removed). Similarly, if a feature is to be lengthened, then this may be based on the priority of the features, e.g., a high priority feature may be lengthened.

These approaches provides mechanisms by which the length of the decision guidance process can be controlled. In particular, the approaches provides examples of how to control a length of a delay of providing guidance and/or a speed at which guidance is provided to the subject and/or caregiver.

The processing system may make use of a machine-learning algorithm or function to control the decision guidance process, such a machine-learning algorithm that has been trained on historic subjects and their decision making processes. The machine-learning algorithm may recommend steps for the processing system to take to modify the decision guidance processes based on such historic information, i.e., based on domain knowledge.

As the subject or caregiver interacts with the decision guidance process, information on the interaction(s) may be logged. For example, time/frequency spent on a particular feature and/or input gathered in questionnaires, can be recorded. This logged information may be used to update the motivational data and/or the health information of the subject. The interaction can be logged using any suitable tagging technology, such as Google® analytics, Adobe® analytics or the like.

Previous embodiments have disclosed approaches in which motivation data alone has been used to control the decision guidance process. However, other embodiments make use of additional data, such as urgency data and/or resource data.

Urgency data v indicates one or more predicted measures of the subject's urgency for receiving future healthcare. The urgency data v may be calculated based on subject health information and/or an urgency indicator from a caregiver. The urgency data may be formed of binary, categorical or numeric data. For instance, the urgency data may be a categorical indicator (e.g., “Low”, “Medium” or “High”) of the urgency of the subject. Thus, the urgency data may be effectively be an urgency state. As another example, the urgency data may be a numeric measure (e.g., on a scale of 0-1, 0-10, 0-100, 1-10 or 1-100, although other scales would be recognized by the skilled person).

If no information on an urgency is available, then the urgency data may be assumed to have a default value (e.g., representing an average level of urgency for a population).

The urgency data v and the motivation data μ may be processed together to produce a conversion readiness level Λ, which indicates a readiness of the subject and/or caregiver to make a healthcare decision.

A conversion readiness level A for a given healthcare solution Si may thereby be derived from the urgency level and motivation level for the particular solution. The user conversion readiness state is high for a given user if both the need for a solution Si is high from a health perspective (based on diagnosis, complain, HCP input) and high from a motivational perspective (user is willing to use the solution Si).

By way of example, if both the urgency data and the motivation data are formed of numeric measures, the conversion readiness level may be obtained by summing, averaging or multiplying the urgency data and the motivation data. As another example, if the urgency data and the motivation data have a categorical data format, the conversion readiness level may represent an average of the two categories (e.g., urgency=“Low” and motivation=“High” average to “Medium”).

The processing system 100 may use the conversion readiness level to control the decision guidance process. In particular, the conversion readiness level may be used to define a recommended decision guidance process, which is used to control the decision guidance process.

The recommended decision guidance process should facilitate an increase in the and/or to increase a motivation (level) of the subject, thereby increasing the conversion readiness level. In some examples, the recommended decision guidance process is configured to facilitate an increase in the accuracy and/or certainty of the urgency data and/or the motivation data, e.g., by controlling the decision guidance process so that additional information on the subject is gained. From a system perspective, if there is insufficient data to accurately evaluate the level of urgency of a patient, the patient conversion readiness level is less accurate than when we have more information about a patient and know from which solution they might benefit.

The recommended decision guidance process should also be based on domain knowledge, e.g., that the use of some features must be prior to the use of other features (based on known decisions making processes) and the impact of a given sequence of features on different patient profiles. Such a recommended decision guidance process may be based on historical data from profiles of similar subjects. For instance, machine-learning techniques can be used to generate the recommended decision guidance process.

It has previously been mentioned how each feature may be associated with a priority level, e.g., indicating an importance of that feature to a successful conversion of the subject. The value of the priority level may depend, for instance, upon the healthcare decision that is being made, a particular solution for the healthcare decision and/or the current conversion readiness level of the subject. The recommended decision guidance process may be controlled further based on the priority of the features in the decision guidance process, e.g., to avoid bypassing high priority features or to prioritize including high priority features in the recommended decision guidance process.

As previously indicated, the priority level(s) of features may be dependent upon the conversion readiness level of the subject. This embodiment recognizes that the importance of a particular feature may depend upon the motivation level and/or urgency level of the subject. For instance, features that recommend a reflection phase are less important for highly motivated subjects than for lowly motivated subjects, meaning that the priority levels can be changed responsive to the conversion readiness of the subject.

It will be appreciated that the priority level may be dependent upon a motivation level and/or urgency level alone (i.e., independent of a conversion readiness level, if available).

In some embodiments, resource data is also used (alongside the motivation data) to control the decision guidance process. This may be in combination with the urgency data or instead of the urgency data.

Resource data may indicate a current usage (e.g., by other subjects) of healthcare solutions, and in particular, healthcare solutions that may result from the decision making process. Resource data may be obtained, for instance, from information provided by healthcare solution providers and/or may be estimated based on historically data.

Control over the decision guidance process may be responsive to an available resource of a predicted solution for the subject. In particular, if the resource data indicates that available resource for a particular solution is low, then the processing system may add delays to the decision guidance process (e.g., using any previously described approach). This reduces the likelihood that a subject will come to a clinical decision (i.e., choose a solution) that cannot be acted upon due to lack of resource, which would significantly impact their motivation and adherence to the solution.

Thus, the use of resource data may supplement motivation data and/or urgency data. This embodiment aims to optimize traffic to increase accuracy of urgency data, but also to increase motivation of subjects to use solutions they might benefit from without overloading healthcare.

FIG. 2 illustrates a method 200 according to an embodiment. The method 200 is designed for controlling a decision guidance process for guiding a subject and/or caregiver in a decision making process for making a healthcare decision for the subject.

The computer-implemented method comprises a step 210 of determining motivation data of the subject, wherein the motivation data indicates one or more predicted measures of the subject's motivation for the subject's future healthcare.

The computer-implemented also comprises a step 220 for controlling the decision guidance process responsive to the determined motivation data of the subject. Approaches for performing steps 210 and 220 have been previously described with reference to the processing system 100 of FIG. 1.

The method 200 may be modified to perform any suitable approach for controlling the decision guidance process (e.g., based on urgency data and/or resource data) previously described.

In particular, the method may comprise a step 230 of providing a user-perceptible output (e.g., a visual representation and/or audio representation) of the decision guidance process and/or a recommended step or stage of the decision guidance process. This may be provided at a user interface, such as a display.

Previously described embodiments propose to adjust a length of the decision guidance process (and/or features of the decision guidance process) based on at least motivation data. However, other embodiments may modify other characteristics of the decision guidance process based on the motivation data.

For instance, the content of the decision guidance process may be modified based on the motivation data. As one example, self-assessment steps may be omitted or included in the decision guidance process responsive to motivation data (e.g., low motivation may include the self-assessment steps and high motivation may exclude the self-assessment steps). As another example, an amount of positive feedback included in the decision guidance process may be responsive to a motivation data (e.g., more positive feedback, e.g., “Good Job!” may be included in the decision guidance process for a low motivated subject whereas, less positive feedback may be included in the decision guidance process for a highly motivated subject, e.g., to avoid appearing patronizing).

As yet another example, more or less educational content may be included in one or more features of the decision guidance process responsive to motivation data.

As yet another example, a user may be given an option to skip a particular feature based on the current motivation level, e.g., the option may be provided for some motivation levels, but not for other motivation levels. For instance, if the decision guidance process includes a motivational feature (e.g., self-reflection on importance of health in life) that is proven to increase motivation of patients doubting that they should act on their health (and is therefore less relevant for motivated users), the system may allow this feature to be skipped for a highly motivated user.

Other suitable mechanisms for controlling a decision guidance process based on motivation data will be apparent to the skilled person.

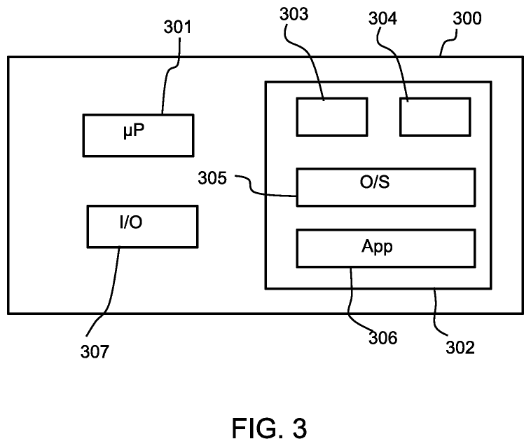

FIG. 3 illustrates a processing system according to an embodiment.

By way of further example, FIG. 3 illustrates an example of a processing system 300 within which one or more parts of an embodiment may be employed. Various operations discussed above may utilize the capabilities of the processing system 300. For example, one or more parts of a system for controlling a decision guidance process may be incorporated in any element, module, application, and/or component discussed herein. In this regard, it is to be understood that system functional blocks can run on a single computer or may be distributed over several computers and locations (e.g., connected via internet).

The processing system 300 includes, but is not limited to, PCs, workstations, laptops, PDAs, palm devices, servers, storages, and the like. Generally, in terms of hardware architecture, the processing system 300 may include one or more processors 301, memory 302, and one or more I/O devices 307 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 301 is a hardware device for executing software that can be stored in the memory 302. The processor 301 can be virtually any custom made or commercially available processor, a central processing unit (CPU), a digital signal processor (DSP), or an auxiliary processor among several processors associated with the processing system 300, and the processor 301 may be a semiconductor based microprocessor (in the form of a microchip) or a microprocessor.

The memory 302 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 302 may incorporate electronic, magnetic, optical, and/or other types of storage media. Note that the memory 302 can have a distributed architecture, where various components are situated remote from one another, but can be accessed by the processor 301.

The software in the memory 302 may include one or more separate programs, each of which comprises an ordered listing of executable instructions for implementing logical functions. The software in the memory 302 includes a suitable operating system (O/S) 305, compiler 304, source code 303, and one or more applications 306 in accordance with exemplary embodiments. As illustrated, the application 306 comprises numerous functional components for implementing the features and operations of the exemplary embodiments. The application 306 of the processing system 300 may represent various applications, computational units, logic, functional units, processes, operations, virtual entities, and/or modules in accordance with exemplary embodiments, but the application 306 is not meant to be a limitation.

The operating system 305 controls the execution of other computer programs, and provides scheduling, input-output control, file and data management, memory management, and communication control and related services. It is contemplated by the inventors that the application 306 for implementing exemplary embodiments may be applicable on all commercially available operating systems.

Application 306 may be a source program, executable program (object code), script, or any other entity comprising a set of instructions to be performed. When a source program, then the program is usually translated via a compiler (such as the compiler 304), assembler, interpreter, or the like, which may or may not be included within the memory 302, so as to operate properly in connection with the O/S 305. Furthermore, the application 306 can be written as an object oriented programming language, which has classes of data and methods, or a procedure programming language, which has routines, subroutines, and/or functions, for example but not limited to, C, C++, C #, Pascal, BASIC, API calls, HTML, XHTML, XML, ASP scripts, JavaScript, FORTRAN, COBOL, Perl, Java, ADA, .NET, and the like.

The I/O devices 307 may include input devices such as, for example but not limited to, a mouse, keyboard, scanner, microphone, camera, etc. Furthermore, the I/O devices 307 may also include output devices, for example but not limited to a printer, display, etc. Finally, the I/O devices 307 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 307 also include components for communicating over various networks, such as the Internet or intranet.

If the processing system 300 is a PC, workstation, intelligent device or the like, the software in the memory 302 may further include a basic input output system (BIOS) (omitted for simplicity). The BIOS is a set of essential software routines that initialize and test hardware at startup, start the O/S 305, and support the transfer of data among the hardware devices. The BIOS is stored in some type of read-only-memory, such as ROM, PROM, EPROM, EEPROM or the like, so that the BIOS can be executed when the processing system 300 is activated.

When the processing system 300 is in operation, the processor 301 is configured to execute software stored within the memory 302, to communicate data to and from the memory 302, and to generally control operations of the processing system 300 pursuant to the software. The application 306 and the O/S 305 are read, in whole or in part, by the processor 301, perhaps buffered within the processor 301, and then executed.

When the application 306 is implemented in software it should be noted that the application 306 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 306 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

It will be understood that disclosed methods are preferably computer-implemented methods. As such, there is also proposed the concept of a computer program comprising code means for implementing any described method when said program is run on a processing system, such as a computer. Thus, different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

1. A computer-implemented method of controlling a decision guidance process for guiding a subject and/or caregiver in a decision making process for making a healthcare decision for the subject, the computer-implemented method comprising:
determining motivation data of the subject, wherein the motivation data indicates one or more predicted measures of the subject's motivation for the subject's future healthcare; and
controlling the decision guidance process responsive to the determined motivation data of the subject.

2. The computer-implemented method of claim 1, wherein the step of controlling the decision guidance process comprises controlling a length of the decision guidance process responsive to the determined motivation data.

3. The computer-implemented method of claim 2, wherein the step of controlling a length of the decision guidance process comprises controlling a length of a delay of guidance to be provided to the subject and/or caregiver.

4. The computer-implemented method of claim 2, wherein the step of controlling a length of the decision guidance process comprises controlling a speed at which guidance is provided to the subject and/or caregiver.

5. The computer-implemented method of claim 1, wherein:
the decision guidance process defines one or more recommended steps for the subject and/or caregiver to take in the decision making process;
the step of controlling the decision guidance process comprises controlling the defining

6. The computer-implemented method of claim 5, wherein the step of controlling the decision guidance process comprises controlling whether or not a step, for the decision making process, is included in the one or more recommended steps.

7. The computer-implemented method of claim 5, wherein the step of controlling the decision guidance process comprises selecting a recommended step based on a predicted length of time for the subject and/or caregiver to carry out the recommended step.

8. The computer-implemented method of claim 1, wherein
the decision guidance process defines a sequence of stages for the decision making process, wherein each stage is able to be completed using one of a respective set of steps; and
the step of controlling the decision guidance process comprises selecting, for each stage, a step from the respective set of steps to be carried out by the subject and/or caregiver.

9. The computer-implemented method of claim 1, further comprising a step of determining urgency data of the subject, wherein the urgency data indicates one or more predicted measures of the subject's urgency for receiving future healthcare; and
wherein the step of controlling the decision guidance process comprises controlling the decision guidance process to be further responsive to the determined urgency data.

10. The computer-implemented method of claim 9, wherein the step of controlling the decision guidance process comprises:
using the motivation data and the urgency data to determine a conversion readiness level, indicating a readiness of the subject and/or caregiver to make a healthcare decision; and
controlling the decision guidance process responsive to the conversion readiness level.

11. The computer-implemented method of claim 1, further comprising a step of determining resource data for the subject, wherein the resource data indicates one or more predicted measures of the availability of a healthcare resource for the subject during the subject's future healthcare;
wherein the step of controlling the decision guidance process comprises controlling the decision guidance process to be further responsive to the determined resource data.

12. The computer-implemented method of claim 1, wherein the step of determining motivation data of the subject comprises:
tracking the actions of the subject and/or caregiver during the decision making process; and
using the tracked actions to determine the motivation data of the subject.

13. The computer-implemented method of claim 12, wherein:
the decision guidance process defines one or more recommended steps for the subject and/or caregiver to take in the decision making process; and
the step of tracking the actions of the subject comprises tracking completion of each recommended step by the subject and/or caregiver.

14. A computer program product comprising computer program code means which, when executed on a computing device having a processing system, cause the processing system to perform all of the steps of the method according to claim 1.

15. A processing system configured to control a decision guidance process for guiding a subject and/or caregiver in a decision making process for making a healthcare decision for the subject, the processing system being configured to:
determine motivation data of the subject, wherein the motivation data indicates one or more predicted measures of the subject's motivation for the subject's future healthcare; and
control the decision guidance process responsive to the determined motivation data of the subject.
